(19) United States
(12) Patent Application Publication
(10) Pub. No.: US 2011/0268970 A1
(43) Pub. Date: Nov. 3, 2011

(54) MESOPOROUS NANOPARTICLES

(75) Inventors: Jackie Y. YING, Singapore (SG); Yu HAN, Singapore (SG)

(73) Assignee: AGENCY FOR SCIENCE, TECHNOLOGY AND RESEARCH, Singapore (SG)

(21) Appl. No.: 13/177,886

(22) Filed: Jul. 7, 2011

Related U.S. Application Data
(62) Division of application No. 11/631,642, filed on Jan. 23, 2008.
(60) Provisional application No. 60/586,082, filed on Jul. 6, 2004.

(51) Int. Cl. B32B 5/16; A61K 9/14; C12N 11/14; A61K 48/00; A61K 47/04; A61P 35/00; A61P 9/12; A61P 29/00; A61P 3/10; A61P 5/00; A61P 31/18; C01B 33/12; B01J 21/08; B01J 31/02; B01J 31/12; A61K 38/02; B82Y 30/00

(52) U.S. Cl. 428/402; 514/1.1; 424/400; 435/176; 514/44 R; 514/770; 423/335; 502/232; 502/150; 502/152; 977/773

(57) ABSTRACT

The present invention provides a process for making mesoporous nanoparticles. The process comprises providing an acidic mixture comprising a fluorocarbon surfactant, a second surfactant and a silica precursor. The silica precursor is then reacted to form the mesoporous nanoparticles.

### TECHNICAL FIELD

The present invention relates to a process for making mesoporous nanoparticles using a fluorocarbon surfactant.

### BACKGROUND OF THE INVENTION

Research on mesoporous materials synthesis has been mainly fodused on mesostructural diversity, compositional flexibility and morphological control. The ability to derive mesoporous particles with a controlled particle size would be important for many practical applications. For example, ultrafine mesoporous particles would be very useful in catalysis and gas adsorption, since they would provide greater pore accessibility and facilitate molecular diffusion. They could also act as the host matrix for the synthesis of quantum dots and magnetic nanoparticles in functional materials and bioimaging applications. Ultrafine mesoporous particles could also act as carriers for drugs, genes and proteins for novel biomedical applications.

Some examples of ultrafine mesoporous particles have been sporadically reported, but the type of mesostructure, the degree of structural ordering and the range of pore sizes have been limited. Aerosol-mediated self-assembly has been used to obtain mesoporous silica spheres with hexagonal and vesicular pore structures, and transition-metal oxide spheres with disordered pore structures, but special equipment is needed for this approach.

One method for synthesizing mesoporous nanoparticles involves the use of a cationic alkylammonium surfactant as a mesostructural template, and a non-ionic triblock copolymer surfactant for suppressing particle growth. A disadvantage with this synthesis is that it required basic conditions, and could not be used in an acidic medium since the triblock copolymer surfactant would co-assemble with silica as a liquid-crystalline mesophase under acidic conditions, and would not then work towards suppressing particle growth. With the restriction of templates usable for basic media (i.e. to alkylammonium surfactants), the mesostructures and pore sizes obtainable by this approach would be limited.

There is therefore a need for a simple process for making nanometer-sized particles with tunable pore sizes.

### OBJECT OF THE INVENTION

It is an object of the present invention to overcome or substantially ameliorate at least one of the above disadvantages. It is a further object to at least partially satisfy the above need.

### SUMMARY OF THE INVENTION

In a first aspect of the invention there is provided a process for making mesoporous nanoparticles comprising:
providing an acidic mixture comprising a fluorocarbon surfactant, a second surfactant and a silica precursor; and
reacting the silica precursor to form the mesoporous nanoparticles.

The acidic mixture may comprise water, and may be an aqueous mixture. It may be a solution, a dispersion or an emulsion, and may be a microemulsion. It may have a pH between about 0.5 and about 5, or between about 1 and about 3. The fluorocarbon surfactant may be anionic, cationic, non-ionic or zwitterionic. The second surfactant may be anionic, cationic, non-ionic or zwitterionic. It may not be a fluorocarbon surfactant. It may be a polymeric surfactant, and may be a copolymer surfactant, for example a block copolymer surfactant. It may be an alkylene oxide block copolymer surfactant, e.g. an EO/PO block copolymer surfactant. The fluorocarbon surfactant and the second surfactant may be miscible or immiscible.

The silica precursor may comprise a hydrolysable silane such as an alkoxysilane. It may comprise for example a trialkoxysilane or a tetraalkoxysilane, or a mixture of the two.

The acidic mixture may also comprise a hydrophobic material. The hydrophobic material may comprise an aromatic, aliphatic or alicyclic hydrocarbon, or a combination of two or more of these, or may comprise some other type of hydrophobic material. It may be a hydrophobic liquid.

The step of preparing the acidic mixture may comprise combining the silica precursor with an acidic surfactant mixture. The acidic surfactant mixture may be aqueous. It may be a solution, a micellar solution, a microemulsion, an emulsion, a dispersion or some other type of mixture. The ratio of silica precursor to acidic surfactant mixture may be between about 1:100 and about 1:2 on a w/w, v/v or w/v basis, and may be about 1:20. Before, during and/or after the combining the mixture may be agitated, e.g. shaken, stirred, swirled, sonicated or otherwise agitated. The acidic surfactant mixture may be prepared by combining the fluorocarbon surfactant with the second surfactant to form a surfactant mixture, and combining (e.g. dissolving, dispersing, emulsifying) the surfactant mixture in an acidic solution. The acidic solution may have a pH between about 0.5 and about 5, or between about 1 and about 3. Alternatively the fluorocarbon surfactant may be combined with the acidic solution to form a fluorocarbon surfactant mixture, and this may be combined with the second surfactant. As a further alternative the second surfactant may be combined with the acidic solution to form a second surfactant mixture, and this may be combined with the fluorocarbon surfactant. Any or all of the above mixtures may be agitated (e.g. shaken, stirred, swirled, sonicated or otherwise agitated). Any or all of the above mixtures may be a solution, a micellar solution, a microemulsion, an emulsion, a dispersion or some other type of mixture.

If the acidic mixture comprises a hydrophobic material, the hydrophobic material may be added at any stage during the process of preparing the acidic mixture. It may be added before, at the same time as or after either or both of the surfactants, or before, at the same time as or after the silica precursor. It may be added with or without agitation.

The process may comprise the step of agitating the acidic mixture to form a solution, a dispersion or an emulsion. The emulsion may be a microemulsion. The agitating may be vigorous, moderate or mild. It may comprise shaking, stirring, sonicating, ultrasonicating, swirling or some other form of agitation. The step of reacting may comprise the step of agitating the acidic mixture or the step of agitating the acidic mixture may be a separate step conducted before the step of reacting.

The step of reacting the silica precursor may comprise hydrolysing and/or condensing the silica precursor to form the mesoporous nanoparticles, which may be mesoporous silica nanoparticles. This step may comprise the steps of:
agitating the acidic mixture for sufficient time and at a sufficient temperature for at least partial hydrolysis of the silica precursor to form a hydrolysate; and
maintaining the mixture, or emulsion, at a temperature and for a time sufficient for reaction of the silica precursor and/or the hydrolysate to form the nanoparticles.

The step of agitating may be conducted at ambient temperature or some other temperature. It may be for example between about 10 and about 80° C., or between about 20 and about 40° C. It may be conducted for between about 5 and about 50 hours or more.

The step of maintaining the mixture may be conducted at between about 70 and about 150° C., and may be between about 80 and 120° C. It may be conducted for between about 10 and 100 hours. During the step of maintaining the mixture may be agitated or it may have no external agitation.

The process may comprise the step of heating from the agitating temperature to the maintaining temperature. The heating may take between about 1 minute and 1 hour.

The ratio between the fluorocarbon surfactant and the second surfactant in the acidic mixture may be between about 1:1 and about 10:1 on a w/w or v/v basis, and may be about 5:4. The concentration of the surfactant (fluorocarbon surfactant plus second surfactant) in the acidic mixture may be between about 0.5 and about 10% on a w/w or w/v basis, and may be about 3%. The concentration of the silica precursor in the acidic mixture may be between about 1 and about 20% on a w/w, w/v or v/v basis, and may be about 5%. The ratio of the fluorocarbon surfactant to the silica precursor may be between about 1:1 and about 1:10 on a w/w or w/v basis, and may be about 1:3.

The process may additionally comprise at least partially separating the nanoparticles from a fluid in which they are located (optionally suspended or dispersed). This may comprise filtering, settling, decanting, centrifuging, vacuum filtering, dialysis, membrane filtering or some other suitable process, and may comprise more than one of these. After the separating, the nanoparticles may be washed with a washing liquid. The washing liquid may be water, or an aqueous liquid, or with a non-aqueous liquid, or an organic liquid, or some combination of these. The particles may be washed once or more than once, and may be washed between 1 and about 10 times or more. Each wash may be with the same washing liquid as any other wash, or may be with a different washing liquid. The washing may comprise exposing the nanoparticles to the washing liquid, e.g. suspending the nanoparticles in the washing liquid, and then separating the nanoparticles from the washing liquid, using any of the separating processes described above. The exposing may be at between about 10 and 100° C., for example about 50° C., and may be for between about 1 minute and 10 hours, for example about 5 hours. It may or may not be accompanied by agitation, for example shaking, stirring, sonicating, ultrasonicating, swirling or some other form of agitation. The process may also comprise heating the nanoparticles. The heating may be to a temperature and for a time sufficient to remove a substantial proportion of the surfactants. The substantial proportion may be greater than about 50%, or greater than about 90%. The temperature may be greater than about 500° C., and may be between about 500 and about 1000° C. The time of heating may be greater than about 1 hour, and may be between about 1 and about 20 hours. It may be about 5 hours. The temperature and time of heating may be sufficient to calcine the nanoparticles. The heating may be in air, or in some other gas, for example, oxygen, nitrogen, carbon dioxide, helium, argon or a mixture of any two or more of these.

In an embodiment there is provided a process for making mesoporous nanoparticles comprising:
combining the silica precursor with an acidic surfactant mixture to form an acidic mixture comprising a fluorocarbon surfactant, a second surfactant and a silica precursor; and
reacting the silica precursor to form the mesoporous nanoparticles.

In another embodiment there is provided a process for making mesoporous nanoparticles comprising:
combining the silica precursor with an aqueous acidic surfactant solution or microemulsion to form an aqueous acidic mixture comprising a fluorocarbon surfactant, a second surfactant and a silica precursor; and
reacting the silica precursor to form the mesoporous nanoparticles.

In another embodiment there is provided a process for making mesoporous nanoparticles comprising:
combining the silica precursor with an acidic surfactant mixture to form an acidic mixture comprising a fluorocarbon surfactant, a second surfactant and a silica precursor;
agitating the acidic mixture for sufficient time and at a sufficient temperature for at least partial hydrolysis of the silica precursor to form a hydrolysate; and
maintaining the mixture, or emulsion, at a temperature and for a time sufficient for reaction of the silica precursor and/or the hydrolysate to form the nanoparticles.

In another embodiment there is provided a process for making mesoporous nanoparticles comprising:
combining a tetraalkoxysilane with an aqueous acidic surfactant solution or microemulsion comprising a fluorocarbon surfactant and a second surfactant, to form an aqueous acidic mixture;
agitating the acidic mixture for sufficient time at between about 25 and about 40° C. for at least partial hydrolysis of the tetraalkoxysilane, to form a solution or microemulsion comprising a hydrolysate of the tetraalkoxysilane; and
maintaining the solution or microemulsion at about 100° C. and for a time sufficient for condensation of the silica precursor and/or the hydrolysate to form the nanoparticles.

In another embodiment there is provided a process for making mesoporous nanoparticles comprising:
combining the silica precursor with an acidic surfactant mixture to form an acidic mixture comprising a fluorocarbon surfactant, a second surfactant and a silica precursor;
agitating the acidic mixture for sufficient time and at a sufficient temperature for at least partial hydrolysis of the silica precursor to form a hydrolysate;
maintaining the mixture, or emulsion, at a temperature and for a time sufficient for reaction of the silica precursor and/or the hydrolysate to form the nanoparticles;
at least partially separating the nanoparticles from a fluid in which they are located; and
washing the nanoparticles with a washing liquid.

In another embodiment there is provided a process for making mesoporous nanoparticles comprising:
combining the silica precursor with an acidic surfactant mixture to form an acidic mixture comprising a fluorocarbon surfactant, a second surfactant and a silica precursor;
agitating the acidic mixture for sufficient time and at a sufficient temperature for at least partial hydrolysis of the silica precursor to form a hydrolysate;
maintaining the mixture, or emulsion, at a temperature and for a time sufficient for reaction of the silica precursor and/or the hydrolysate to form the nanoparticles;
at least partially separating the nanoparticles from a fluid in which they are located; and
heating the nanoparticles to a temperature and for a time sufficient to remove a substantial proportion of the surfactants.

In another embodiment there is provided a process for making mesoporous nanoparticles comprising:
combining a tetraalkoxysilane with an aqueous acidic surfactant solution or microemulsion comprising a fluorocarbon surfactant and a second surfactant, to form an aqueous acidic mixture;
agitating the aqueous acidic mixture for sufficient time at between about 25 and about 40° C. for at least partial hydrolysis of the tetraalkoxysilane, to form a solution or microemulsion comprising a hydrolysate of the tetraalkoxysilane;
maintaining the solution or microemulsion at about 100° C. and for a time sufficient for condensation of the silica precursor and/or the hydrolysate to form the nanoparticles;
at least partially separating the nanoparticles from a fluid in which they are located; and
heating the nanoparticles to a temperature and for a time sufficient to remove a substantial proportion of the surfactants.

The invention also provides mesoporous nanoparticles when made by the process of the first aspect.

In a second aspect of the invention there is provided a mesoporous nanoparticle baying a particle size between about 1 and about 500 nm, or between about 50 and about 300 nm, and a mean pore size between about 1 and about 50 am, or between about 5 and about 30 nm or greater than 10 nm, or between about 10 and 50 nm. The nanoparticles may have a 3-D cubic or 3-D foam-like mesostructure, or may have a 2-D hexagonal or wormlike mesostructure. The mesoporous nanoparticle may comprise silica, and may comprise mesoporous silica. The silica may be doped with other elements, for example titanium, aluminium or zirconium. The mesoporous nanoparticle may be spherical or some other regular shape. There is also provided a plurality of mesoporous nanoparticles as described above. The mean particle size of the nanoparticles may be between about 1 and about 500 nm. The particle size distribution may be broad or narrow. There may be less than about 50% of nanoparticles having a particle size more than 10% different from (greater than or less than) the mean particle size. The mesoporous nanoparticle(s) may be made by the process of the first aspect of the invention.

In a third aspect of the invention there is provided a use of a mesoporous nanoparticle, or a plurality thereof, according to the invention for an application, selected from the group consisting of catalysis, gas adsorption, synthesis of quantum dots and magnetic nanoparticles in functional materials and bioimaging applications, and as carriers for drugs, genes and proteins for novel biomedical applications. There is also provided a mesoporous nanoparticle, or a plurality thereof, when used in an application selected from the group consisting of catalysis, gas adsorption, synthesis of quantum dots and magnetic nanoparticles in functional materials and bioimaging applications, and as carriers for drugs, genes and proteins for novel biomedical applications.

In a fourth aspect of the invention there is provided a catalyst comprising a mesoporous nanoparticle, or a plurality thereof, according to the present invention, said nanoparticle having a catalytic species associated therewith. The catalytic species may be adsorbed or bound or sorbed on and/or in the nanoparticle. The catalytic species may be an organic catalytic species, an organometallic catalytic species or an inorganic catalytic species. It may be an enzyme or some other catalytic species. It may be covalently boned to the nanoparticle or it may be associated in some other fashion.

There is also provided a process for making a catalyst according to the fourth aspect comprising exposing a mesoporous nanoparticle, or a plurality thereof, according to the present invention, to the catalytic species. The catalytic species may be in solution, for example an aqueous or non-aqueous solution. The exposing may comprise agitating the nanoparticle(s) and the catalyst. The agitating may comprise mixing, shaking; stirring, sonicating, ultrasonicating, swirling or some other form of agitation. The agitation may be continued for sufficient time to allow the catalyst to become associated with the nanoparticle(s). Alternatively the process may comprise passing the catalyst or the solution past the nanoparticle(s), for example through a nanoparticle bed (comprising a plurality of the nanoparticles). The process may comprise application of pressure, for example greater than about 10 MPa, e.g. between about 25 and 50 MPa.

In a fifth aspect of the invention there is provided a nanoparticle, or a plurality thereof, according to the invention, said nanoparticle having a drug and/or a gene and/or a protein associated (e.g. adsorbed or bound or sorbed) therewith. The drug and/or gene and/or protein may be reversibly associated with the nanoparticle, or may be irreversibly associated therewith.

There is also provided a process for making a nanoparticle, or a plurality thereof, According to the fifth aspect comprising exposing a mesoporous nanoparticle, or a plurality thereof, according to the present invention, to the drug and/or gene and/or protein. The drug and/or gene and/or protein may be in solution or in emulsion, microemulsion or suspension. The exposing may comprise agitating the nanoparticle(s) and the catalyst. The agitating may comprise mixing, shaking, stirring, sonicating, ultrasonicating, swirling or some other form of agitation. The agitation may be continued for sufficient time to allow the drug and/or gene and/or protein to become associated with the nanoparticle(s). Alternatively the process may comprise passing the drug and/or gene and/or protein past the nanoparticle(s), for example through a nanoparticle bed (comprising a plurality of the nanoparticles). The process may comprise application of pressure, for example greater than about 10 MPa, e.g. between about 25 and 50 MPa.

In a sixth aspect of the invention there is provided a method for catalysing a reaction of a starting material to a product, or for producing the product, comprising exposing the starting material to a catalyst according to the fourth aspect of the invention, wherein the catalytic species of the reaction is capable of catalysing the reaction. The starting material may be in solution, which may be an aqueous or a non-aqueous solution. The non-aqueous solution may be a solution in organic solvent (e.g. an alcohol, an ether, an ester, a hydrocarbon, a halocarbon or some other solvent). The method may comprise agitating the starting material or the solution and the catalyst. The agitating may comprise mixing, shaking, stirring, sonicating, ultrasonicating, swirling or some other form of agitation. The agitation may be continued for sufficient time to allow starting material to be converted to the product. Alternatively the method may comprise passing the starting material or the solution past the catalyst, for example through a catalyst bed comprising a plurality of catalysts (i.e. nanoparticles having a catalytic species associated therewith). The catalyst bed may be of suitable dimensions so that the residence time of the starting material in the bed is sufficient to allow it to be converted to the product.

In a seventh aspect of the invention there is provided a product when made by the method of the sixth aspect of the reaction.

In an eighth aspect of the invention there is provided a method for delivering a drug and/or a gene and/or a protein comprising exposing a nanoparticle according to the if aspect of the invention to an environment in which the drug and/or gene and/or protein is released from the nanoparticle. The environment may be the body of a patient, whereby the method is a method for delivering the drug and/or gene and/or protein to the patient. The environment may be an aqueous environment or some other environment. The method may be for treatment of a condition, e.g. a disease, in the patient, whereby the so drug and/or gene and/or protein for the condition. The patient may be a human patient, or may be a non-human patient. The patient may be a vertebrate, and the vertebrate May be a mammal, a marsupial or a reptile. The mammal may be a primate or non-human primate or other non-human mammal. The mammal may be selected from the group consisting of human, non-human primate, equine, murine, bovine, leporine, ovine, caprine, feline and canine. The mammal may be selected from a human, horse, cattle, cow, bull, ox, buffalo, sheep, dog, cat, goat, llama, rabbit, ape, monkey and a camel, for example. The condition may be for example cancer, AIDS, arthritis, diabetes, hormonal disfunction, hypertension, pain or some other condition.

In a ninth aspect of the invention there is provided the use of a nanoparticle according to the fifth aspect of the invention for the manufacture of a medicament for the treatment of a condition, e.g. a disease. The condition may be for example cancer, AIDS, arthritis, diabetes, hormonal disfunction, hypertension, pain or some other condition.

In a tenth aspect of the invention there is provided a medicament comprising a nanoparticle (or a plurality thereof) according to the fifth aspect of the invention, optionally together with one or more clinically acceptable additives, carriers and/or excipients.

In an eleventh aspect of the invention there is provided a method for treating a condition, e.g. a disease, in a patient comprising administering to the patient a therapeutic quantity of a medicament according to the tenth aspect of the invention, or of nanoparticles according to the fifth aspect of the invention. The administering may be orally, topically, by injection (intravenous, intramuscular etc.), by inhalation or by some other appropriate route.

### BRIEF DESCRIPTION OF THE DRAWINGS

A preferred form of the present invention will now be described by way of example with reference to the accompanying drawings wherein:

FIG. 1 shows experimental results for calcined IBN-1 according to the present invention: a) SEM micrograph (inset: TEM micrograph); b)-d) HR-TEM micrographs taken at different incidences: [100.], [110] and [111], respectively (inset: the corresponding FT patterns); e) XRD pattern; f) N₂ adsorption-desorption isotherm;

FIG. 2 shows experimental results for calcined IBN-2 according to the present invention: a) SEM micrograph (inset: N₂ adsorption-desorption isotherm); b)-d) HR-TEM micrographs taken at different incidences; [100.], [211] and [110], respectively (inset: corresponding FT patterns);

FIG. 3 shows experimental results for calcined IBN-3 according to the present invention: a) SEM micrograph (inset: TEM micrograph); b) HR-TEM micrograph; c) N2 adsorption-desorption isotherm;

FIG. 4 shows experimental results for calcined IBN-4 according to the present invention: a) SEM micrograph (inset: TEM micrograph); b) HR-TEM micrograph; c) XRD pattern; d) N₂ adsorption-desorption isotherm;

FIG. 5 shows experimental results for surfactant-extracted IBN-5 according to the present invention: a) SEM micrograph (inset: HR-TEM micrograph); b) XRD pattern; c) N₂ adsorption-desorption isotherm; d) 29Si MAS NMR spectrum; e) 13C CP/MAS NMR spectrum.

FIG. 6 shows experimental results for TEM micrograph of an IBN-2 nanoparticle according to the present invention, along the [110] direction, marked to show the twins of ccp phase, and the intergrowth of hcp phase in this small particle.

### DETAILED DESCRIPTION OF THE PREFERRED EMBODIMENTS

In one form, the present invention provides a simple wet-chemical process that enables the synthesis of nanometer-sized particles (50-300 nm) with tunable pore sizes in the range of 5-30 nm. This fluorocarbon surfactant-mediated synthesis may be is generalized to achieve various pore structures, including 3-D cubic Im-3m, 3-D cubic Fm-3m, 2-D hexagonal p6m, foam-like and worm-like pores, as well as different material compositions. The synthesis may be capable of producing ultrafine particles with well-defined mesopores, regular particle morphology and excellent pore accessibility. The mesopores may be adjustable in size and may have high structural ordering. The process uses two different types of surfactant. The inventors propose that the fluorocarbon surfactant may be used to control the growth of the mesoporous particles, whereas the second surfactant may act as a supramolecular template for formation of the periodic mesostructure.

The process comprises providing an acidic mixture comprising a fluorocarbon surfactant, a second surfactant and a silica precursor, and reacting the silica precursor to form the mesoporous nanoparticles.

The acidic mixture may comprise water, and may be an aqueous mixture. It may comprise one or more other additives, for example salts. It may be a solution, a dispersion or an emulsion, and may be a microemulsion. If it is an emulsion, or a microemulsion, it may have a mean droplet size between about 1 and about 500 nm, or between about 1 and 200, 1 and 100, 1 and 50, 1 and 20, 10 and 500, 100 and 500, 250 and 500, 10 and 200, 10 and 100, 50 and 200, 20 and 100 or 50 and 300 nm, and may have a mean droplet size of about 1, 2, 3, 4, 5, 10, 15, 20, 25, 30, 35, 40, 45, 50, 60, 70, 80, 90, 100, 150, 200, 250, 300, 350, 400, 450 or 500 nm. It may have a pH between about 0.5 and about 5, or between about 0.5 and 2, 0.5 and 1, 1 and 5, 2 and 5, 2 and 4, 1 and 2 or 1 and about 3. It may have a pH about 0.5, 1, 1.1, 1.2, 1.3, 1.4, 1.5, 1.6, 1.7, 1.8, 1.9, 2, 2.1, 2.2, 2.3, 2.4, 2.5, 3, 3.5, 4, 4.5 or 5. The fluorocarbon surfactant may be anionic, cationic, non-ionic or zwitterionic. It may comprise perfluoroether groups (e.g. —OCF(CF₃)CF₂O—). It may be a quaternary ammonium surfactant. It may comprise some non-fluorinated groups, e.g. alkyl groups. It may be for example FC4 [(C₃F₇O(CFCF₃CF₂O)₂CFCF₃CONH(CH₂)₃N⁺(C₂H₅)₂CH₃I⁻)]. The second surfactant may be anionic, cationic, non-ionic or zwitterionic. It may not be a fluorocarbon surfactant. It may be a polymeric surfactant, and may be a copolymer surfactant. The copolymer surfactant may be a block copolymer, or may be a random copolymer, an alternating copolymer or some other type of copolymer. The block copolymer may be a diblock, triblock or other copolymer. It may have between 2 and 5 blocks or more than 5 blocks. It may have an odd or an even number of blocks, and may have 2, 3, 4 or 5 blocks. It may is have hydrophilic blocks alternating with hydrophobic blocks. The terminal blocks may be hydrophobic, or may be hydrophilic, or one may be hydrophilic and one hydrophobic. The copolymer surfactant may have 2, 3, 4, 5 or more than 5 different types of blocks (i.e. different monomers). It may be an alkylene oxide block copolymer surfactant. It may be an EO/PO copolymer surfactant, e.g. an EO/PO block copolymer surfactant. Suitable second surfactants include Pluronic P65 (EO20PO30EO20), Pluronic P85 (EO26PO40EO26), Pluronic 25R4, Pluronic F108 (EO129PO56EO129), Pluronic P123 (EO20PO70EO20) and Pluronic F127 (EO97PO69EO97). The fluorocarbon surfactant and the second surfactant may be miscible or immiscible, or may be partially miscible.

The silica precursor may be a hydrolysable silane such as an alkoxysilane. It may be for example a trialkoxysilane or a tetraalkoxysilane, or a mixture of the two. Alternatively it may be an alkanoxysilane (e.g. acetoxysilane), oximosilane (e.g. butanone oximo silane), amidosilane (e.g. benzamidosilane), enoloxysilane (e.g. propen-2-yloxysilane) or some other suitable silane. Suitable silanes include, but are not restricted to tri- and tetra-alkoxysilanes such as tetramethoxysilane (TMOS), tetraethoxysilane (TEOS), tetrabutoxysilane (TBOS), tetrapropoxysilane (TPOS), methyltrimethoxysilane (MTMS), methyltriethoxysilane (MTES), ethyltriethoxysilane (ETES), octyltriethoxysilane (OTES), octyltrimethoxysilane (OTMS), hexadecyltrimethoxisilane (HDTMS) and hexadecyltriethoxisilane (HDTES), octadecyltrimethoxysilane (ODTMS), octadecyltriethoxyisilane (ODTES) as well as methyl polysilicate (MPS), ethyl polysilicate (EPS), polydiethoxysilane (PDES), hexamethyl disilicate, hexaethyl disilicate or functional trialkoxysilanes (eg methacryloyloxypropyltrimethoxysilane, phenyltriethoxysilane (PTES), phenyltrimethoxysilane (PTMS), glycidoxypropoxyltrimethoxysilane (GLYMO), glycidoxypropyltriethoxysilane (GLYEO), mercaptopropyltriethoxysilane (MATES), mercaptopropyltrimethoxysilane (MPTMS), aminopropyltrimethoxysilane (APTMS), aminopropyltriethoxysilane (APTES), 3-(2-aminoethylamino)propyltrimethoxysilane (DATMS), 3-[2-(2-aminoethylamino)ethylamino]propyltrimethoxysilane (TATMS), [2-(cyclohexenyl)ethyl]triethoxysilane (CHEETES), vinyltrimethoxysilane (VTMS), vinyltriethoxysilane (VTES). Other silica precursors that may be used include partial hydrolysates of any of the above or of mixtures of any two or more of any of the above, including dimers, mixed dimers, trimers, mixed trimers etc. Bis(trialkoxysilyl) alkanes, such as 1,2-bis(trimethoxysilyl)ethane, or 1,2-bis(triethoxysilyl)ethane may also be used. It will be understood that mixtures of the abovementioned precursors may be used in any is desired combination. These mixtures may be used to tailor the properties of the nanoparticles.

The acidic mixture may also comprise a hydrophobic material. The hydrophobic material may be an aromatic, aliphatic or alicyclic hydrocarbon, or may be some other type of hydrophobic material. The hydrophobic material may be a hydrophobic liquid. It may be a swelling agent. The hydrophobic liquid may be an organic liquid. It may be aromatic or aliphatic, or it may be a halo compound or some other hydrophobic liquid. Suitable aliphatic liquids include aliphatic hydrocarbons of between about 6 and about 20 carbon atoms, and the aliphatic hydrocarbons may be branched or straight chain. The aliphatic liquid may be a mixture of aliphatic hydrocarbons. The aliphatic hydrocarbons may have between 6 and 20, 6 and 18, 6 and 16, 6 and 12, 8 and 20, 12 and 20, 16, and 20, 8 and 16 or 10 and 18 carbon atoms, and may have 6, 8, 10, 12, 14, 16, 18 or 20 carbon atoms. Suitable aromatic liquids include toluene, xylene, 1,3,5-trimethylbenzene (TMB), ethylbenzene, diethylbenzene, cumene or a mixture of aromatic liquids. The aromatic liquid may have between about 6 and about 20 carbon atoms, or between 6 and 18, 6 and 16, 6 and 12, 8 and 20, 12 and 20, 16 and 20, 8 and 16 or 10 and 18 carbon atoms, and may have 6, 7, 8, 9, 10, 11, 12, 13, 14, 15, 16, 17, 18, 19 or 20 carbon atoms. It may comprise a mixture of hydrophobic compounds. The boiling point of the hydrophobic material may be greater than the temperature for reacting the silica precursor. It may be greater than about 80, 90, 100 or 110° C., and may be about 80, 90, 100, 110, 120, 130, 140, 150, 160, 170, 180, 190 or 200° C., or it may be greater than 200° C.

The step of preparing the acidic mixture may comprise combining the silica precursor with an acidic surfactant mixture. The acidic surfactant mixture may be a solution, a micellar solution, a microemulsion, an emulsion, a dispersion or some other type of mixture. The ratio of silica precursor to acidic surfactant mixture may be between about 1:100 and about 1:2 on a w/w, v/v or w/v basis, and may be between about 1:100 and 1:5, 1:100 and 1:10, 1:100 and 1:20, 1:100 and 1:50, 1:50 and 1:5, 1:20 and 1:5, 1:10 and 1:5, 1:50 and 1:10, 1:30 and 1:10, 1:25 and 1:15, 1:22 and 1:18, and may be about 1:100, 1:50, 1:40, 1:35, 1:30, 1:25, 1:24, 1:23, 1:22, 1:21, 1:20, 1:19, 1:18, 1:17, 1:16, 1:15, 1:10 or 1:5. The acidic surfactant mixture may be prepared by combining the fluorocarbon surfactant with the second surfactant to form a surfactant mixture, and combining (e.g. dissolving, dispersing, emulsifying) the surfactant mixture in an acidic solution. The acidic solution may have a pH between about 0.5 and about 5, or between about 0.5 and 2, 0.5 and 1, 1 and 5, 2 and 5, 2 and 4, 1 and 2 or 1 and about 3. It may have a pH about 0.5, 1, 1.1, 1.2, 1.3, 1.4, 1.5, 1.6, 1.7, 1.8, 1.9, 2, 2.1, 2.2, 2.3, 2.4, 2.5, 3, 3.5, 4, 4.5 or 5. Alternatively the fluorocarbon surfactant may be combined with the acidic solution to form a fluorocarbon surfactant mixture, and this may be combined with the second surfactant. As a further alternative the second surfactant may be combined with the acidic solution to form a second surfactant mixture, and this may be combined with the fluorocarbon surfactant.

The process may comprise the step of agitating the acidic mixture to form a solution, a dispersion or an emulsion. The emulsion may be a microemulsion. The agitating may be vigorous, moderate or mild. It may comprise mixing, shaking, stirring, sonicating, ultrasonicating, swirling or some other form of agitation. The step of reacting may comprise the step of agitating the acidic mixture or the step of agitating the acidic mixture may be a separate step conducted before the step of reacting.

The step of reacting the silica precursor may comprise hydrolysing and/or condensing the silica precursor to form the mesoporous nanoparticles, which may be mesoporous silica nanoparticles. This step may comprise the steps of
agitating the acidic mixture for sufficient time and at a sufficient temperature for at least partial hydrolysis of the silica precursor to form a hydrolysate; and
maintaining the mixture, or emulsion, at a temperature and for a time sufficient for reaction of the silica precursor and/or the hydrolysate to form the nanoparticles.

The step of agitating may be conducted at ambient temperature or some other temperature. It may be for example between about 10 and about 80° C., or between about 10 and 60, 10 and 40, 10 and 20, 20 and 80, 40 and 80, 20 and 60, 20 and 40, 15 and 30 or 15 and 25° C., and may be at about 10, 15, 20, 25, 30, 35, 40, 45, 50, 55, 60, 65, 70, 75 or 80° C. It may be conducted for between about 5 and about 50 hours or more than 50 hours, and may be conducted for between about 5 and 40, 5 and 30, 5 and 20, 5 and 10, 10 and to 50, 20 and 50, 10 and 40, 10 and 30, 15 and 25 or 17 and 23 hours, and may be for about 5, 10, 15, 16, 17, 18, 19, 20, 21, 22, 23, 24, 25, 30, 35, 40, 45 or 50 hours or more than 50 hours.

The step of maintaining the mixture may be conducted at between about 70 and about 150° C., and may be between about 70 and 130, 70 and 100, 100 and 150, 120 and 150, 80 and 120, 90 and 110 or 95 and 105° C., and may be at about 70, 75, 80, 85, 90, 95, 100, 105, 110, 120, 125, 130, 135, 140, 145 or 150° C. It may be conducted for between about 10 and 100 hours, or between about 10 and 50, 10 and 30, 20 and 100, 50 and 100, and 50, 15 and 30, 20 and 28 or 22 and 26 hours, and may be for about 10, 12, 16, 20, 21, 22, 23, 24, 25, 26, 27, 28, 29, 30, 32, 36, 42, 48, 60, 72, 84, 96 or 100 hours, or may be for more than 100 hours. During the step of maintaining the mixture may be agitated or it may have no external agitation. It may be mildly or vigorously agitated, and may be swirled, stirred, shaken or otherwise agitated. It will be understood that heating to the maintaining temperature may cause mild agitation due to thermal currents in the mixture.

The process may comprise the step of beating from the agitating temperature to the maintaining temperature. The heating may take between about 1 minute and about 1 hour, or between about 1 and 30 minutes, or 1 and 20, 1 and 10, 1 and 5, 5 and 60, 5 and 30, 10 and 50, 125 and 45, 10 and 30, 30 and 50 or 10 and 20 minutes, and may take about 1, 2, 3, 4, 5, 6, 7,8,9 10, 15, 20, 25, 30, 35, 40, 45, 50, 55 or 60 minutes.

The ratio between the fluorocarbon surfactant and the second surfactant in the acidic mixture may be between about 1:1 and about 10:1 on a w/w or v/v basis, or may be between about 1:1 and 5:1, 1:1 and 3:1, 1:1 and 2:1, 1:1 and 1.5:1, 1:1 and 1.25:1, 2:1 and 10:1, 5:1 and 101, 2:1 and 5:1, 1.05:1 and 1.5:1, 1.1:1 and 1.5:1, 1.2:1 and 1.4:1, 1.2:1 and 1.3:1 or 1.1:1 and 1.3:1 and may be about 5:4, 1:1, 2:1, 3:1, 4:1, 5:1, 6:1, 7:1, 8:1 9:1, 10:1, 1.05:1, 1.1:1, 1.15:1, 1.2:1, 1.25:1, 1.3:1, 1.35:1, 1.4:1, 1.45:1, 1.5:1, 1.6:1, 1.7:1, 1.8:1 or 1.9:1. The concentration of the surfactant (fluorocarbon surfactant plus second surfactant) in the acidic mixture may be between about 0.5 and about 10% on a w/w or w/v basis, or may be between about 1 and 10, 2 and 10, 5 and 10, 0.5 and 5, 0.5 and 2, 1 and 5, 2 and 5, 2 and 4 or 2.5 and 3.5% and may be about 0.5, 1, 1.5, 2, 2.5, 3, 3.5, 4, 4.5 or 5%. The concentration of the silica precursor in the acidic mixture may be between about 1 and about 20% on a w/w, w/v or v/v basis, or may be between about 1 and 10, 1 and 5, 1 and 2, 2 and 10, 5 and 10, 10 and 20, 15 and 20, 10 and 15, 2 and 8, 3 and 7 or 4 and 6% and may be about 1, 2, 3, 3.5, 4, 4.5, 5, 5.5, 6, 6.5, 7, 7.5, 8, 9, 10, 11, 12, 13, 14, 1,5 16, 17, 18, 19 or 20%. The ratio of the fluorocarbon surfactant to the silica precursor to may be between about 1:1 and about 1:10 on a w/w or w/v basis, and may be between about 1:1 and 1:5, 1:1 and 1:2, 1.2 and 1:10, 1:5 and 1:10, 1:2 and 1:5 or 1:2 and 1:4, and may be about 1:1, 1:1.5, 1:2, 1:2.5, 1:2.6, 1:2, 7, 1:2.8, 1:2.9, 1:3, 1:3.1, 1:3.2, 1:3.3, 1:3.4, 1:3.5, 1:4, 1:4.5, 1:5, 1:6, 1:7, 1:8, 1:9 or 1:10.

The process may additionally comprise at least partially separating the nano particles is from a fluid in which they are located. The nanoparticles may be suspended or dispersed in the fluid. The separating may comprise filtering, settling, decanting, centrifuging, vacuum filtering, dialysis, membrane filtering, extraction or some other suitable process, and may comprise more than one of these, which may be conducted simultaneously or sequentially. After the separating, the nanoparticles may be washed with a washing liquid. The washing liquid may be water, or an aqueous liquid, or with a non-aqueous liquid, or an organic liquid, or some combination of these. It may be for example an alcohol, such as ethanol, methanol, propanol, isopropanol; or it may be some other common solvent, e.g. a ketone, an ester, a chloroalkane, or a mixture of any two or more of these. An example of a suitable washing liquid is acidified ethanol, e.g. ethanol with aqueous hydrochloric acid added. The particles may be washed once or more than, once, and may be washed between 1 and about 10 times or more, i.e. 1, 2, 3, 4, 5, 6, 7, 8, 9, 10 or more than 10 times. Each wash may be with the same washing liquid as any other wash, or may be with a different washing liquid. The washing may comprise exposing the nanoparticles to the washing liquid, e.g. suspending the nanoparticles in the washing liquid, and then separating the nanoparticles from the washing liquid, using any of the separating processes described above. The exposing may be at between about 10 and about 100° C., or between about 10 and 50, 10 and 30, 10 and 20, 20 and 100, 50 and 100, 80 and 100, and 80, 30 and 70 or 40 and 60° C., and may be at about 10, 20, 30, 40, 50, 60, 70, 80, 90 or 100° C. It may be for between about 1 minute and 10 hours, and may be for between about 1 and 10 hours, 5 and 10 hours, 1 and 5 hours, 2 and 8 hours, 3 and 7 hours, 4 and 6 hours, 1 and 60 minutes, or, 1 and 30, 1 and 10, 10 and 60 or 30 and 60 minutes, or between about 30 minutes and 10 hours, 30 minutes and 5 hours or 30 minutes and 2 hours, and may be for about 1, 2, 3, 4, 5, 6, 7, 8, 9, 10, 15, 20, 25, 30, 40 or 50 minutes, or about 1, 1.5, 2, 2.5, 3, 3, 5, 4, 4.5, 5, 5.5, 6, 6.5, 7, 7.5, 8, 8.5, 9, 9.5 or 10 hours or may be for more than 10 hours. Each step of washing, independently, may or may not be accompanied by agitation, for example shaking, stirring, sonicating, ultrasonicating, swirling or some other form of agitation. The process may comprise heating the nanoparticles to a temperature and for a time sufficient to remove a substantial proportion of the surfactants. The substantial proportion may be greater than about 50%, or greater than about 55, 60, 65, 70, 75, 80, 85, 90 or 95%, and may be about 50, 55, 60, 65, 70, 75, 80, 85, 90, 95, 98, 99, 99.5, 99.9 or 100%. The temperature may be greater than about 500° C., or greater than about 600, 700, 800 or 900° C., and may be between about 500 and about 1000° C., or between about 500 and 800, 500 and 600, 520 and 580, 530 and 570, is 540 and 560, 600 and 1000, 800 and 1000 or 600 and 800° C., and may be at about 500, 510, 520, 530, 540, 550, 560, 570, 580, 590, 600, 650, 700, 750, 800, 850, 900, 950 or 1000° C. or greater than 1000° C. The time of heating may be greater than about 1 hour, or greater than 2, 3, 4, 5 or 10 hours, and may be between about 1 and about 20 hours, or between about 1 and 10, 1 and 5, 5 and 20, 10 and 20, 15 and 20, 2 and 8, 3 and 7 or 4 and 6 hours. It may be about 1, 2, 3, 4, 5, 6, 7, 8, 9, 10, 11, 12, 13, 1,4 15, 16, 17, 18, 19 or 20 hours. The temperature and time may be sufficient to calcine the nanoparticles. The heating may be in air, or in some other gas, for example, oxygen, nitrogen, carbon dioxide, helium, argon or a mixture of any two or more of these.

The process may also comprise drying the nanoparticles. The drying may be as conducted before the heating to remove surfactants, and may be conducted after the step of washing the nanoparticles, or after any or all of the individual steps of washing, if the nanoparticles are washed more than once. Thus after the formation of the nanoparticles by reaction of the silica precursor and/or hydrolysate thereof, the particles may be separated from a fluid in which they are located. They may be then washed, or may be washed and so then dried, or may be washed and then heated to remove surfactants, or may be washed, then dried, then heated to remove surfactants, or they may be dried and then heated to remove surfactants, or they may be heated to remove surfactants. The step of drying may comprise heating the nanoparticles. The heating may be to a temperature between about 30 and 150° C., or between about 30 and 100, 30 and 50, 50 and 150, 100 and 150, 50 and 100 or 80 and 120° C., and may be to about 30, 40, 50, 60, 70, 80, 90, 100, 110, 120, 130, 140 or 150° C., or may be to greater than 150° C. The heating may be in air, nitrogen, argon, helium, carbon dioxide or some other gas or a mixture of any two or more; of these. Alternatively or additionally the step of drying may comprise freeze-drying. The step of drying may additionally or alternatively comprise passing a stream of gas over and/or through the particles. The gas may be a gas that is inert to the particles, and may be for example air, nitrogen, argon, helium, carbon dioxide or a mixture of these, and may be dried. The step of drying may additionally or alternatively comprise applying a partial vacuum to the nanoparticles. The partial vacuum may have an absolute pressure of for example between about 0.01 and 0.5 atmospheres, or between about 0.01 and 0.1, 0.01 and 0.05, 0.1 and 0.5, 0.25 and 0.5, 0.05 and 0.1 or 0.1 and 0.25 atmospheres, and may have an absolute pressure of about 0.01, 0.05, 0.1, 0.2, 0.3, 0.4 or 0.5 atmospheres. The drying may comprise vacuum drying or freeze drying.

In one representative process according to the present invention, an alkoxysilane (e.g. tetraalkoxysilane) is added to an aqueous acidic surfactant mixture comprising a fluorocarbon surfactant and a second surfactant. The mixture may be a solution, a micellar solution or a microemulsion of the fluorocarbon surfactant and the second surfactant. The mixture may optionally also comprise a hydrophobic material, which may be capable of assisting in formation of pores in the final nanoparticles. On addition of the alkoxysilane, optionally with agitation over time, the alkoxysilane at least partially hydrolyses in the acidic medium to form a hydrolysate. This typically takes place at slightly above ambient temperatures, but may be conducted at lower temperatures for a longer time. The hydrolysate may be a partial hydrolysate, that is not all of the alkoxy groups may be hydrolysed, or it may be a complete hydrolysate in which all of the alkoxy groups have been hydrolysed to silanol groups. The hydrolysate may be water soluble, so that the resulting aqueous acidic mixture may be a solution, or it may be a microemulsion. If a hydrophobic material is present, it may be located in the dispersed phase of the microemulsion. The aqueous acidic mixture is then heated to an elevated temperature sufficient to promote condensation of the hydrolysate to form the nanoparticles of the invention. This temperature is typically around 100° C., however it will be understood that lower temperatures may be used for longer times, or higher temperatures for shorter times, so long as the conditions of temperature and pressure are such that the mixture does not boil. At the elevated temperature, condensation of the hydrolysate, optionally together with any unreacted alkoxysilane, to form the mesoporous nanoparticles of the invention. These may have shape, nanoporosity and size which is controlled by the nature and quantity of the surfactants, the hydrophobic material (if present) and the alkoxysilane. The mesoporous nanoparticles may then be separated, e.g. by centrifuging, or by solvent extraction, and then dried. The surfactants may be at least partially removed, either by washing with a solvent, such as ethanol, or by calcining the nanoparticles at high temperature.

The invention also provides a mesoporous nanoparticle having a particle size between about 1 and about 500 nm. The particle size may be between about 1 and 400, 1 and 300, 1 and 200, 1 and 100, 1 and 50, 1 and 20, 10 and 500, 50 and 500, 100 and 500, 200 and 500, 300 and 500, 50 and 400, 50 and 300, 100 and 300, 200 and 300 or 100 and 200 nm. The particle may have a mean pore size greater than about 1 nm, or greater than about 2, 3, 4, 5, 6, 7, 8, 9, 10, 11, 12, 13, 14, 15, 16, 17, 18, 19 or 20 nm, or between about 1 and about 50 nm, or between about 1 and 40, 1 and 30, 1 and 20, 1 and 10, 1 and 5, 5 and 20, 5 and 10, 10 and 20, 10 and 50, 20 and 50, 30 and 50, 10 and 40 or 20 and 30 nm, and may have a mean pore size about 1, 2, 3, 4, 5, 5.2, 5.5, 5.8, 6, 6.4, 6.5, 7, 7.5, 8, 8.5, 9, 9.5, 10, 11, 12, 13, 14, 15, 16, 17, 18, 19, 19.5, 20, 21, 22, 23, 24, 25, 30, 35, 40, 45 or 50 nm or greater than 50 nm. The mesoporous nanoparticle may comprise silica, and may comprise mesoporous silica. The silica may be doped with one or more other elements, for example titanium, aluminium or zirconium. The mesoporous nanoparticle may be spherical or some other regular shape. The invention also provides a plurality of mesoporous nanoparticles as described above. The mean particle size of the nanoparticles may be between about 1 and about 500 nm. The mean particle size may be between about 1 and 400, 1 and 300, 1 and 200, 1 and 100, 1 and 50, 1 and 20, 10 and 500, 50 and 500, 100 and 500, 200 and 500, 300 and 500, 50 and 400, 50 and 300, 100 and 300, 200 and 300 or 100 and 200 nm. The particle size distribution may be broad or narrow. There may be less than about 50% of nanoparticles having a particle size more than 10% different from (greater than or less than) the mean particle size, or less than about 40, 30, 20 or 10% of nanoparticles having a particles size more than 10, 15, 20, 25, 30, 35, 40, 45 or 50% different from the mean particle size, or may have about 50, 45, 40, 35, 30, 25, 20, 25, 10 or 5% of nanoparticles within that size range.

The surface area of the particle(s), e.g. BET surface area, maybe between about 200 and about 2000 m²/g, and may be between about 500 and 2000, 1000 and 2000, 1500 and 2000, 200 and 1000, 200 and 500, 1000 and 1500, 500 and 1000, 500 and 600, 700 and 700, 700 and 800, 800 and 900, 900 and 1000, 500 and 900, 700 and 900 or 700 and 850 m²/g, and may be about 200, 300, 400, 500, 525, 550, 575, 600, 625, 650, 675, 700, 710, 720, 730, 740, 750, 760, 770, 780, 790, 800, 810, 820, 830, 840, 850, 875, 900, 950, 1000, 1100, 1200, 1300, 1400, 1500, 1600, 1700, 1800, 1900 or 2000 m²/g. The pore volume may be between about 0.2 and about 2 cm³/g, or between about 0.5 and 2, 1 and 2, 0.2 and 1, 0.2 and 0.5, 0.5 and 1, 0.5 and 0.75 or 0.75 and 1 cm³/g, and may be about 0.1, 0.2, 0.3, 0.4, 0.5, 0.54, 0.55, 0.6, 0.65, 0.7, 0.73, 0.75, 0.8, 0.82, 0.85, 0.88, 0.9, 0.95, 1, 1.1, 1.2, 1.3, 1.4, 1.5, 1.6, 1.7, 1.8, 1.9 or 2 cm³/g.

The particles may be round or spherical, or may be oblate spherical, rod-like, aggregated, ellipsoid, ovoid, a modified oval shape, dome shaped, hemispherical; a round ended cylinder, capsule shaped, discoid, prismatic, acicular or polyhedral (either regular or irregular) such as a cube, a rectangular prism, a rectangular parallelepiped, a triangular prism, a hexagonal prism, rhomboid or a polyhedron with between 4 and 60 or more faces, or it may be some other shape, for example an irregular shape. By contrast with nanoparticles reported in the literature, in which the pore structures were limited to 2-D hexagonal shapes, and the pore sizes were limited to less than about 8 nm, the mesoporous nanoparticles of the present invention may have 3-D cubic or 3-D foam-like mesostructures, or may have a 2-D hexagonal or wormlike mesostructure. Mesostructure refers to how the pores are arranged in the nanoparticles. The nanoparticles may have large pore sizes (diameters), for example greater than 10 nm.

The particles of the present invention may be used for an application selected from the group consisting of catalysis, gas adsorption, synthesis of quantum dots and magnetic nanoparticles in functional materials and bioimaging applications, and as carriers for drugs, genes and proteins for novel biomedical applications. Thus the high and controllable pore surface area makes them suitable for adsorption of gases, or catalyst species, or biological species, such as drugs, enzymes etc. for delivering them to the site of action.

Thus the particle of the present invention may be converted into a catalyst by having a catalytic species associated with the particle. The catalytic species may be adsorbed or bound or sorbed on and/or in the nanoparticle. The catalyst may be used for so catalysing a reaction by exposing a starting material, optionally in solution, to the catalyst, whereby the catalytic species of the catalyst is capable of catalysing the reaction of the starting material to a product. The catalytic species may be a biocatalyst, for example an enzyme, and the reaction may be a biocatalyst-catalysed (e.g. enzyme catalysed) reaction. The pore size of the mesoporous nanoparticles of the present invention may match the dimensions of an enzyme, which may allow the enzyme to be encapsulated in the pores of the nanoparticles with long-term stability.

### Example

### Experimental

IBN-1: 0.65 g of Pluronic F127 and 0.8 g of FC-4 were dissolved in 40 nil of HCl solution (0.02 M), followed by the introduction of 2.2 g of tetraethyl orthosilicate (TEOS). The solution was stirred at 30° C. for 20 h, and then transferred to an autoclave for further condensation at 100° C. for 1 day.

IBN-2: 0.25 g of Pluronic F127 and 0.7 g of FC-4 were dissolved in 30 ml of HCl solution (0.02 M), followed by the introduction of 0.25 g of TMB. After stirring for 2 h, 1.5 g of TEOS were added. The solution was stirred at 30° C. for 20 h, and then transferred to an autoclave for further condensation at 100° C. for 1 day.

IBN-3: 0.25 g of Pluronic P65 and 0.7 g of FC-4 were dissolved in 35 ml of HCl solution (0.02 M), followed by the introduction of 0.75 g of TMB. After stirring for 2 h, 2.0 g of TEOS were added. The solution was stirred at 25° C. for 20 h, and then transferred to an autoclave for further condensation at 100° C. for 1 day.

IBN-4; 0.25 g of Pluronic P123 and 0.7 g of FC-4 were dissolved in 40 rill of HCl solution (0.02 M), followed by the introduction of 1.0 g of TEOS. The solution was stirred at 30° C. for 20 h, and then transferred to an autoclave for further condensation at 100° C. for 1 day.

IBN-5: 0.5 g of Pluronic F108 and 0.7 g of FC-4 were dissolved in 30 ml of HCl solution (0.02 M), followed by the introduction of 1.4 g of 1,2-bis(trimethoxysilyl)ethane. The solution was stirred at 37° C. for 20 h, and then transferred to an autoclave for further condensation at 100° C. for 1 day.

Except for IBN-5, the as-synthesized materials were collected by centrifuge, dried in air and calcined at 550° C. for 5 h for surfactant removal. In IBN-5, surfactants were removed by extraction; 0.5 g of the as-synthesized sample was treated twice in 100 ml of ethanol with 2 g of 2 M HCl solution at 50° C. for 5 h.

XRD patterns were obtained with a Siemens D5005 diffractometer using Cu Kα radiation. SEM micrographs were obtained on a JEOL JSM-6700F electron microscope. TEM experiments were performed on a JEOL JEM-3010 electron microscope with an acceleration voltage of 300 kV. The nitrogen sorption isotherms were obtained using a Micromeritics ASAP 2020M system; the samples were degassed for 10 h at 150° C. before the measurements. ²⁹Si and ¹³C CP/MAS NMR spectra were taken with a Bruker AV500WB system with a 4-mm DVT CP/MAS probe; chemical shifts for both spectra were referenced to trimethylsilane (TMS) at 0 ppm.

### Results and Discussion

The syntheses were carried out in a weakly acidic medium (pH=1.6-1.8), where a homogeneous solution was formed through mixing a soluble silica precursor, a non-ionic triblock copolymer surfactant ((ethylene oxide)ₓ-(propylene oxide)_{y}-(ethylene oxide)ₓ), and a cationic fluorocarbon surfactant FC-4 (C₃F₇O(CFCF₃CF₂O)₂CFCF₃CONH(CH₂)₃N⁺C₂H₅)₂CH₃I⁻). In some cases, organic swelling agent 1,3,5-trimethylbenzene (TMS) was also added to adjust the pore size or vary the mesostructure. Amphiphilic triblock copolymers are capable of self-assembly into micelles with long-range order in aqueous solution, and may act as supramolecular is templates for creating well-ordered mesostructured materials. Fluorocarbon surfactants, however, are not suitable templates for preparing ordered mesoporous materials since the fluorocarbon chains are rigid and lack affinity for each other. They result in the formation of micelles with small aggregation number, instead of periodic long-range order. Also, unlike the hydrocarbon chains of common surfactants (which are hydrophobic but lipophilic), the fluorocarbon chains are hydrophobic and lipophobic. Therefore, hydrocarbon and fluorocarbon surfactants are either immiscible or only partially miscible under most conditions.

The synthetic strategy used in the present work was based on the different properties of these two types of surfactants. The triblock copolymer surfactant would act as the supramolecular template for the periodic mesostructure, whereas the fluorocarbon surfactant would be used to control the growth of mesoporous particles. The process could be described as follows: the weak acidic condition would promote, a slow hydrolysis of silica precursors, and the hydrolyzed silica species would co-assemble with triblock copolymer surfactants to form well-defined mesophases, whose structures and pore sizes would depend on the type of copolymer and the amount/type of organic additives. Simultaneously, fluorocarbon surfactants would surround the silica particles through S⁺X⁻I⁺ interactions with the surface species of the latter, thereby limiting the growth of silica particles. By this approach, five different mesoporous structures were successfully derived with nanometer particle sizes (denoted as IBN-1 to IBN-5 in Table 1).

TABLE 1
Mesoporous nanoparticles obtained with the fluorocarbon
surfactant-mediated synthesis.*

| | | | BET | | |
|---|---|---|---|---|---|
| | | | Surface | Pore | |
| | | | Area | Volume | Pore Size |
| Sample | Mesostructure | Template | (m²/g) | (cm³/g) | (nm)^{†} |
| IBN-1 | 3-D Cubic | F127 | 779 | 0.73 | 5.8 |
| | (Im-3m) | | | | |
| IBN-2 | 3-D Cubic | F127 + TMB | 804 | 0.65 | 9.5 |
| | (Fm-3m) | | | | |
| IBN-3 | Mesocellular | P65 + TMB | 821 | 0.82 | 19.5 |
| | Foam | | | | |
| IBN-4 | 2-D Hexagonal | P123 | 709 | 0.88 | 6.4 |
| | (p6m) | | | | |
| IBN-5 | Worm-like | F108 | 575 | 0.54 | 5.2 |

*Fluorocarbon surfactant FC-4 was used in all syntheses to limit the particle size.
^{†}Calculated from the adsorption branch of the N₂ sorption isotherm using the BJH method.

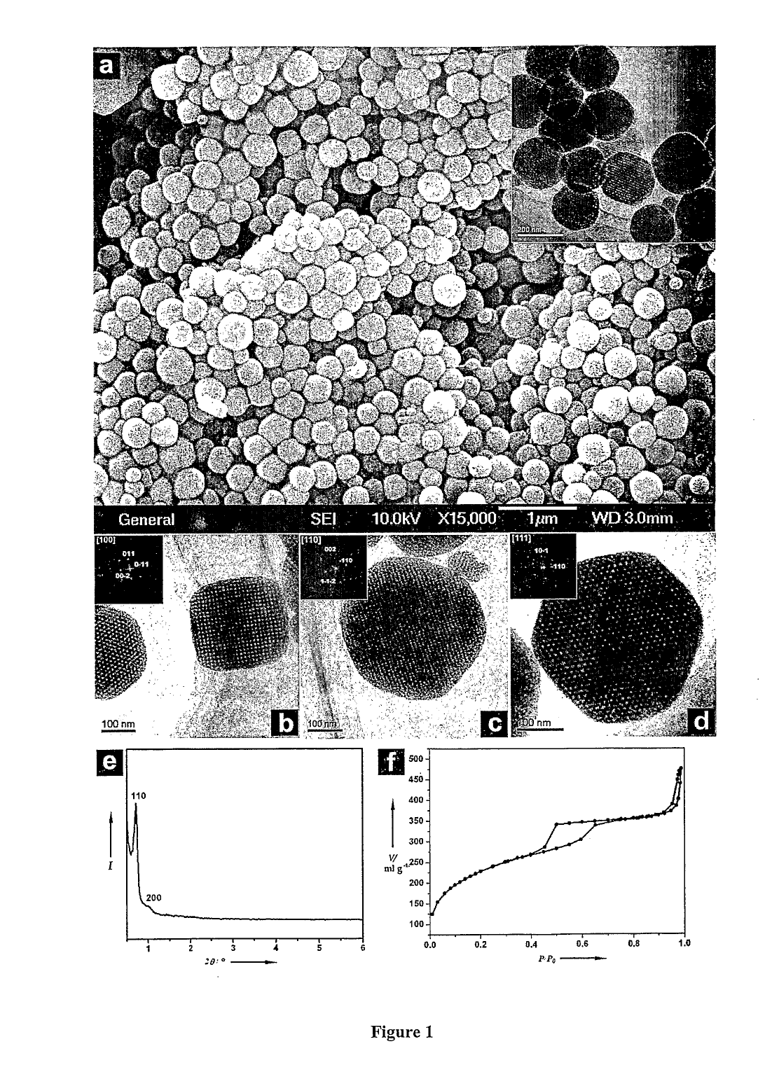

FIG. 1a shows the scanning electron microscopy (SEM) image of calcined IBN-1 that was prepared with Pluronic F127 triblock copolymer (EO₁₀₆PO₇₀EO₁₀₆) and to fluorocarbon surfactant FC-4 using the synthetic approach described above. IBN-1 was composed of relatively uniform particles of 100-300 nm. Transmission electron microscopy (TEM) image (FIG. 1a inset) revealed that these particles were well-dispersed with little aggregation. The XRD pattern of calcined IBN-1 (FIG. 1e) showed two well-resolved peaks with d spacings of 116 Å and 82 Å, respectively, which could be indexed as the 110 and 200 diffractions of a cubic symmetry with a lattice constant α of 164 Å. The high-resolution TEM (HR-TEM) micrographs of this material taken at [100], [110] and [111] incidences and the corresponding Fourier-transforms (FT) are shown in FIGS. 1b, 1c and 1d, respectively. IBN-1 particles displayed morphologies that were in good accordance with their cubic symmetry (for example, square and hexagonal particle morphologies were observed in [100] and [111] directions, respectively). The highly ordered arrangement of mesopores could be observed over the entire particle in all cases, indicating the high quality of the sample. The reflections in the FT patterns could be indexed as 110, 200, 211 and 220 of a cubic phase (Im-3m space group) with; a large lattice constant α of 165 Å, as consistent with the XRD finding. IBN-1 has a Type IV N₂ adsorption-desorption isotherm with a type-H₂ hysteresis loop (FIG. 1f), suggesting that the mesopores were cage-like. The average pore diameter was calculated to be 5.8 nm from the adsorption branch of the isotherm using the Barrett-Joyner-Halenda (BJH) method. Besides the well-defined mesopores, this material showed interparticle (textural) porosity (as evidenced by the adsorption step at high relative pressures of >0.9), which constituted a quarter of the total pore volume of 0.73 cm³/g. IBN-1 has a high Brunauer-Emmett-Teller (BET) surface area of 779 m²/g.

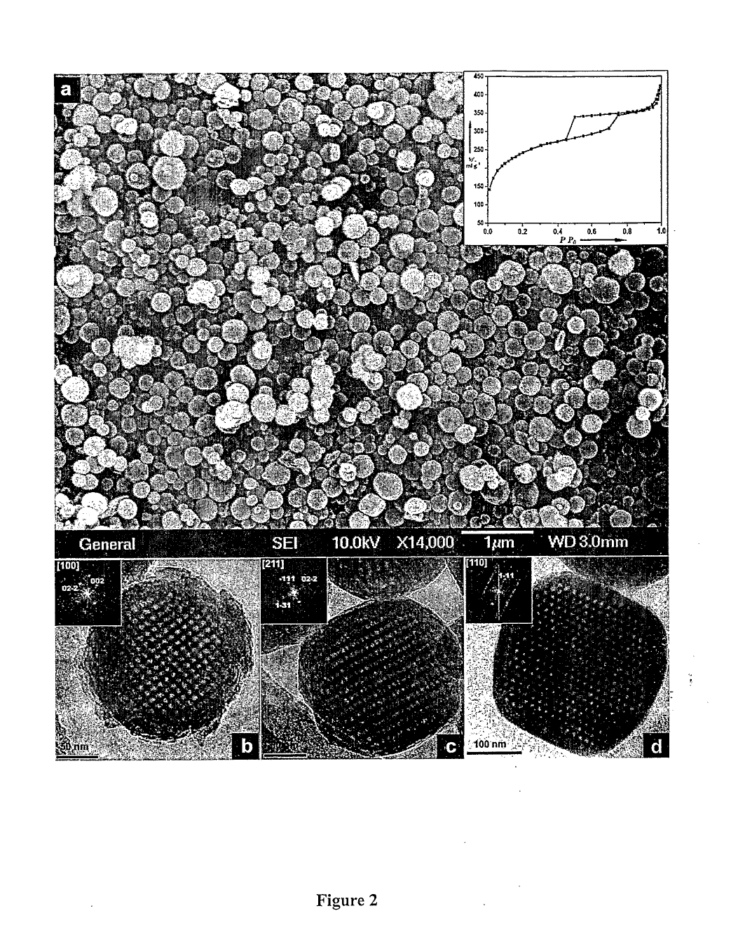

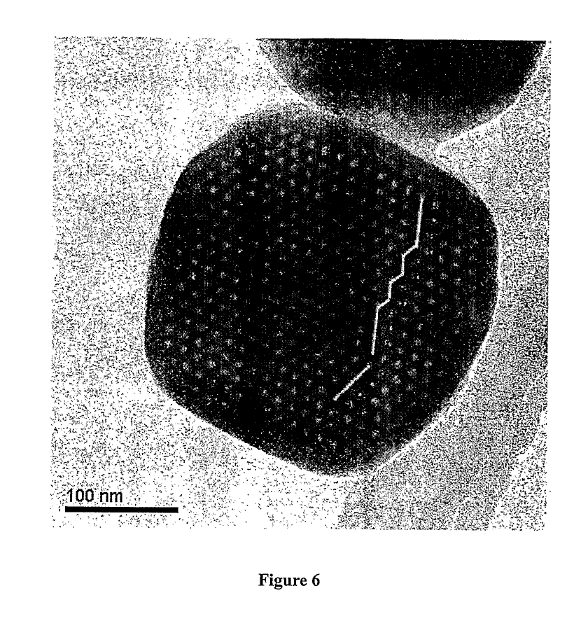

IBN-2 was synthesized under conditions similar to that of IBN-1, except that a large amount of TMB was added (see Table 1). It was composed of well-dispersed particles of 50-300 nm (FIG. 2a). N₂ sorption isotherm (FIG. 2a inset) showed that IBN-2 possessed cage-type pores averaging 9.5 nm, which was much larger than that of IBN-1 due to the addition of TMB swelling agent. HR-TEM micrographs taken at various incidences (FIGS. 2b-d) illustrated the well-ordered large pores in IBN-2. The spots in FT patterns (FIGS. 2b-d insets) were indexed as 111, 200, 220, 311 and 222 reflections for a cubic system with a large lattice constant α of 220 Å. The conditions for these reflections were summarized as {hkl: h+k, k+l, l+h even}, {0kl: k, l even}, {hhl: h+l even}, and {00l: l even}. According to extinction rules and previous studies,^{[25,26]}IBN-2 could be assigned to a face-centered cubic structure (Fm-3m). Notably, the FT pattern of [110] incidence showed strong diffuse streaks along [1-11] direction, suggesting the presence of mixed phases. This was confirmed by the corresponding TEM image (FIG. 2d), which illustrated narrow cubic close-packed (ccp, ABCABC . . . ) bands with periodicity in twin relation. In addition, some 3-D hexagonal domains with the cages arranged in hexagonal close-packed (hcp, ABAB . . . ) mode were also observed between the cubic twins as a transitional phase (see FIG. 6). This is the first report of a perfect intergrowth of cubic and 3-D hexagonal phases in such a small particle; similar ingrowth has been observed in large particles of mesoporous silica, such as FDU-1^{[25]} and SBA-12,^{[26]}

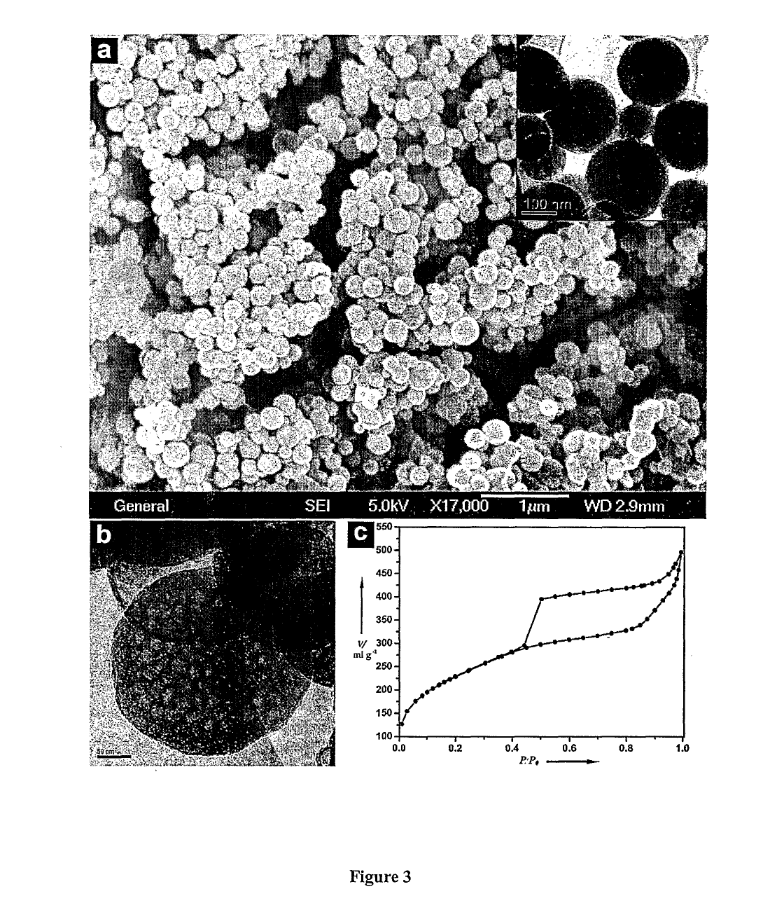

Mesocellular foam (MCF) is a novel mesostructured silica material templated by oil-in-water microemulsions. The ultralarge mesopores (25-40 nm) have made MCF particularly useful as catalyst supports and separation media for processes involving large substrates. Conventional MCF has a cauliflower-type morphology with a particle size of tens of microns. Using the present fluorocarbon surfactant-mediated synthesis, spherical nanoparticles of MCF (50-300 nm) were successfully obtained as IBN-3 (FIG. 3a). In this synthesis, Pluronic P65 triblock copolymer (EO₂₀PO₃₀EO₂₀) and TMB were used as the surfactant and oil, respectively, for the formation of microemulsion template. The ultralarge foam-like pores in the particles obtained could be easily seen with TEM even at relatively low magnification (FIG. 3a inset). The HR-TEM micrograph showed that the pores were ˜20 nm in diameter (FIG. 3b), as consistent with the average adsorption BJH pore size (19.5 nm) (Table 1). The pore diameter of IBN-3 could be tailored in the range of 15-30 nm without changing the particle size and morphology, by varying the amount of TMB added in the synthesis.

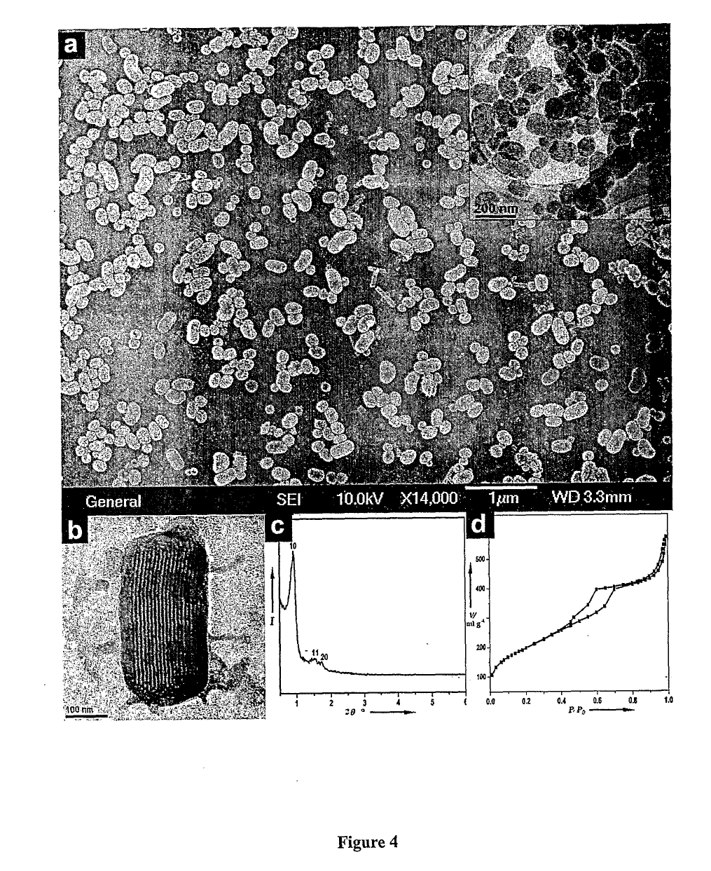

IBN-1, IBN-2 and IBN-3 all possessed cage-type mesopores, as evidenced by the type-H₂ hysteresis loops in their sorption isotherms. The fluorocarbon surfactant-mediated synthesis could also be used to derive nanoparticles with channel-like mesopores. For example, IBN-4, which was templated by Pluronic P123 (EO₂₀PO₇₀EO₂₀), exhibited a mesostructure typical of a 2-1) hexagonal phase (p6m) with a lattice constant α of 105 Å (FIGS. 4b and 4c). IBN-4 showed channel-type mesopores with a uniform diameter of 6.4 nm, as calculated from the N₂ sorption isotherm, which has a type-H₁ hysteresis loop (FIG. 4d). Most of the IBN-4 particles have a rod-like morphology (200-400 nm long and 50-150 nm wide) (FIG. 4a), in good accordance with their 2-D hexagonal mesostructure.

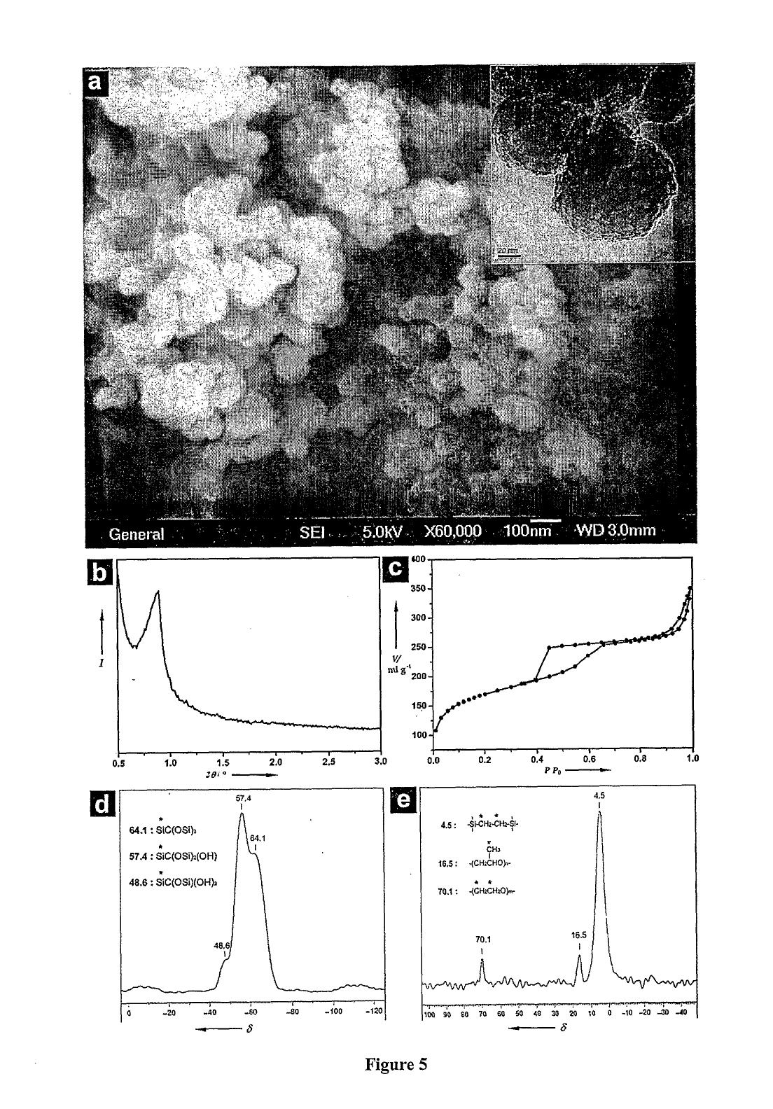

Periodic mesoporous organosilicas (PMOs), synthesized from organosilanes (R′O)₃Si—R—Si(OR′)₃, were reported independently in 1999 by three research groups. The organic groups and inorganic silicon species were alternately distributed within the framework of PMOs, which allowed their mechanical strength, hydrophilicity and surface properties to be tuned by varying the type of organic groups incorporated. In this work, organosilanes (1,2-bis(trimethoxysilyl)ethane) and F108 (EO₁₃₂PO₅₀EO₁₃₂) were employed as the precursor and surfactant template, respectively, in our fluorocarbon surfactant-mediated synthesis to prepare nanoparticles of PMO. The surfactant template was removed by ethanol extraction to give IBN-5. The ²⁹Si MAS and ¹³C CP/MAS nuclear magnetic resonance (NMR) spectra (FIGS. 5d and 5e, respectively) showed that all of the Si atoms in the material were bonded covalently to C atoms, and the framework consisted of SiO_{1.5}—CH₂—CH₂—SiO_{1.5} structural units. It should be noted that in FIG. 5e, the two small peaks at 16.5 ppm and 70.1 ppm were due to C species from the residual triblock copolymer surfactant. Both SEM and TEM micrographs (FIG. 5a and inset) showed that IBN-5 consisted of fairly uniform particles of ˜100 nm. However, unlike the pure silica materials discussed earlier, IBN-5 nanoparticles were not well-dispersed. The mesopores in IBN-5 could be observed by TEM, but the contrast was relatively weak (FIG. 5a inset) due to the disordered pore arrangement. Only one peak appeared in the XRD pattern (FIG. 5b), further indicating the lack of long-range order in IBN-5. Nevertheless, the pore size distribution in IBN-5 (centered at ˜5.2 nm) was still narrow, as illustrated by the sharp step (at P/P₀˜0.6) in the adsorption isotherm (FIG. 5c). The second adsorption step at high relative pressures of ≧0.9 indicated the presence of substantial textural porosity, and revealed that the interparticle voids were still accessible despite the particle agglomeration.

In summary, the inventors have synthesized nanoparticles with five types of mesostructures. These included the mesoporous silicas with 3-D cubic Im-3m, 3-D cubic Fm-3m, 2-D hexagonal p6m and MCF mesostructures, and the mesoporous organosilica with a disordered worm-like mesostructure. Fluorocarbon surfactant was used in all syntheses. Without the use of a fluorocarbon surfactant it is expected that large, irregular particles would be obtained instead of well-defined nanoparticles. The optimal concentration of FC-4 was 2.0-2.5 wt %. In addition, a mildly acidic condition (pH=1.6-1.8) was necessary for the syntheses. Stronger acidity is expected to promote: a rapid, uncontrolled condensation of silica species, which would not allow for the formation of ultrafine particles with regular morphology. Moreover, the nature and the concentration of the triblock copolymer surfactant were important. It should be noted that under certain conditions, hydrocarbon surfactant and fluorocarbon surfactant could be miscible and form mixed micelles. This should be avoided in the present syntheses, since the fluorocarbon surfactant would be involved in the mixed micelles, instead of being used to suppress the particle growth. In general, the longer its hydrophobic PO segment, the more solubilizing power the triblock copolymer has, and therefore, the more likely it would form mixed micelles with fluorocarbon surfactant. Therefore, the triblock copolymers with relatively long hydrophilic EC) segments, e.g. F127 (EO₁₀₆PO₇₀EO₁₀₆) and F108 (EO₁₃₂PO₅₀EO₁₃₂), were preferred templates for this synthetic strategy, as they could be used over a relatively wide range of concentrations (0.5-3 wt %). These triblock copolymers were used in the synthesis of IBN-1 and IBN-5, respectively. In contrast, the triblock copolymers with low BO/PO ratios, for example Pluronic P123 (EO₂₀PO₇₀EO₂₀), have to be used at very low concentrations (0.5-1 wt %) in the synthesis of 11′W-4, or large particles with an irregular morphology would be obtained. This was possibly because P123 would involve most of FC-4 molecules to form mixed micelles at relatively high concentrations, but when its concentration was kept low, FC-4 molecules would still function towards controlling particle growth. In the cases that involved TMB addition (e.g. IBN-2 and IBN-3), low concentrations of triblock copolymer should also be used in preparing nanoparticles since TMB would increase the hydrophobic volume of the copolymer micelles and consequently increase the tendency of forming mixed micelles with FC-4.

Compared to the previous work on forming small mesoporous particles, the present fluorocarbon surfactant-mediated synthesis has at least three distinct benefits. First, this it, approach could be generally applied for the production of different mesostructures, pore types and material compositions. Various mesostructures could be obtained in the form of nanoparticles by changing the triblock copolymer surfactant, and a high degree of structural ordering was successfully attained. It is also worth mentioning that ultrafine mesoporous organosilicas have rarely been reported, and that Im-3m, Fm-3m and foam-like mesostructures have not been derived as nanoparticles prior to this work. Secondly, the pore sizes could be tuned over a wide range from 5 nm to 30 nm in this generalized synthesis, whereas most of the previous reports have a pore size limitation of ≦5 nm. Lastly, the present method was based on a simple sol-gel process modification, and required no special apparatus for forming nanoparticles.

Also, the present fluorocarbon surfactant-mediated method may be generalized for the synthesis of a variety of mesostructures, as illustrated in the present specification. It has been shown to work under acidic conditions, and may be extended to basic conditions so long as a suitable combination of fluorocarbon surfactant and templating surfactant is employed.

1-31. (canceled)

32. Mesoporous nanoparticles when made by a process comprising:
providing an acidic mixture comprising a fluorocarbon surfactant, a second surfactant and a silica precursor; and
reacting the silica precursor to form the mesoporous nanoparticles.

33. The mesoporous nanoparticles of claim 32, each having a particle size between about 1 and about 500 nm and a mean pore size between about 1 and about 50 nm.

34. The mesoporous nanoparticles of claim 33 wherein the pore size is between about 10 and about 50 nm.

35. The mesoporous nanoparticles of claim 32, each having a particle size between about 32 and about 500 nm and having a mesostructure selected from the group consisting of 3-D cubic, 3-D foam-like, 2-D hexagonal and wormlike.

36. The mesoporous nanoparticle of claim 35 wherein the pore size is greater than about 10 nm.

37. Mesoporous nanoparticles according to claim 32, when used in an application selected from the group consisting of catalysis, gas adsorption, synthesis of quantum dots and magnetic nanoparticles in functional materials and bioimaging applications, and as carriers for drugs, genes and proteins for biomedical applications.

38. Mesoporous nanoparticles according to claim 32, said nanoparticles having a species selected from the group consisting of a drug, a gene and a protein associated therewith.

39. Mesoporous nanoparticles according to claim 32 which are spherical and/or round ended cylinders.

40. Mesoporous nanoparticles according to claim 32 comprising silica doped with another element.

41. Mesoporous nanoparticles according to claim 32 having a catalytic species adsorbed or bound or sorbed on and/or in the nanoparticles.

42. Mesoporous nanoparticles according to claim 41 wherein the catalytic species is selected from the group consisting of an organic catalytic species, an organometallic catalytic species, an inorganic catalytic species and an enzyme.

43. Mesoporous nanoparticles according to claim 32 having a drug and/or a gene and/or a protein associated therewith.

44. Regular shaped mesoporous nanoparticles having a particle size between about 32 and about 500 nm and having a mesostructure selected from the group consisting of 3-D cubic, 3-D foam-like, 2-D hexagonal and wormlike.

45. Nanoparticles according to claim 44 which are spherical and/or round ended cylinders.

46. Nanoparticles according to claim 44 having a pore size between about 10 and about 50 nm.

47. Nanoparticles according to claim 44, said nanoparticles having a species selected from the group consisting of a drug, a gene and a protein associated therewith.

48. Nanoparticles according to claim 44, having a catalytic species adsorbed or bound or sorbed on and/or in the nanoparticle.

49. Nanoparticles according to claim 48 wherein the catalytic species is selected from the group consisting of an organic catalytic species, an organometallic catalytic species, an inorganic catalytic species and an enzyme.
